(19) United States
(12) Patent Application Publication
(10) Pub. No.: US 2012/0082689 A1
(43) Pub. Date: Apr. 5, 2012

(54) Method of treatment an oncological disease

(76) Inventor: Lev Tovberg, Hadera (IL)

(21) Appl. No.: 13/135,693

(22) Filed: Jul. 14, 2011

Related U.S. Application Data
(60) Provisional application No. 61/404,213, filed on Sep. 30, 2010.

(51) Int. Cl. A61K 39/00; A61P 35/00

(52) U.S. Cl. 424/184.1

(57) ABSTRACT

Method of cancer treatment with direct contact between proteins secreted by cancer tumor and patient's blood T-lymphocytes including the stage of weakening during some time humoral system of immunity by medical preparation, in particular by immunodepressant against this system, without weakening cellular system of immunity.

### BACKGROUND OF THE INVENTION

The invention relates to medicine, and namely to its special field—oncology, i.e. methods and means of treatment oncologicl diseases (cancer).

For more than a century the mankind carries out systematic and targeted fight with one of the most dangerous diseases—cancer of different organs (disease of different organs known even from the time of Hippocrates).

On the basis of the general progress of medicine a vast number of attempts have been made to treat cancer, and in many cases positive results have been obtained (up to complete cure, but with inevitable side effects—complications).

Widely distributed opinion that cancer patients destiny is doomed and treatment is hopeless is gradually being overcome.

But up to now a universal method for treating or approach to treating this disease has not been found. However, from ancient times up to the present , the mankind has stored a lot of approaches and medicinal means for fighting separate kinds of cancer—cancer of definite organs,—see, e.g. “Popular medical encyclopedia”, Moscow, Soviet Encyclopedia, 1987, p. 421-424.

In many countries, France for example, all malignant tumors irrespective of their tissue origin are called “cancer”. In the former USSR and other countries only malignant tumors developed from epithelium were called by the term “cancer”. Sometimes tumor is named according to the organ from which it originates or according to its specific part.

Malignant tumors more often appear with elderly people. However, children also manifest new formations from embryonal tissues, nervous tissues, lymphatic tissues, kidney tumors and new vascular formations.

Sickness rate of cancer diseases is not the same in different countries that is connected with their historic, geographical and climatic characteristics as well as with household and hygienic habits of peoples living in these countries.

Considerable growth of cancer diseases in all economically developed countries in the 20th century is connected with increase in atmospheric air pollution, with tumorigenic (cancerogenic) substances having effluents from heating systems and industrial enterprises as their sources, especially exhaust gases of internal-combustion engines (motorcars, aviation).

Considerable role belongs to inhalation of tobacco fumes. Liver primary cancer which occurs very often in Africa and South-Eastern Asia, depends on malnutrition and, in particular, on some tumorigenic substances entering food.

Epidemiological researches permitted to determine some causes of human cancer and, in particular, have found the importance of some chemical substances and irradiations (e.g. radiation).

However, multiple experimental researches have played the most important role.

So, many researches have determined that tumors can be caused by different factors—chemical, physical, biological. That's why multifactor (polyethiological) concept of tumor origin is generally accepted today, having appeared instead of the old theories.

Besides two main ways of cancer prophylaxis (primary prophylaxis and secondary—clinical prophylaxis), problem of early cancer diagnostics acquires a great role. And this is not so simple.

It is considered that early stages of cancer have no symptoms. But it is not exactly this way. Weak manifestations of the disease exist practically always which may give a suspicion.

Each form of malignant tumor manifests not only in ways inherent only to that kind of disease, but there are also symptoms characteristic to tumors in general.

Tumor diagnostics is carried out by the same methods which are used for identifying other human diseases: patient's questionnaires , examinations, different analyses, special methods of investigation (research).

But it is considered that up to the present time medicine is unable to diagnose cancer on the basis of only one laboratory investigation.

Exception is made only to blood diseases as blood test can play a decisive role in diagnostics of leucosis because the system of blood formation itself serves as a hotbed for tumor development process.

Outer forms of tumors are the easiest to identify. They can be observed visually and are easy for taking materials for morphological investigation under the microscope (biopsy).

Early diagnostics of inner organ tumor is the most difficult. In this case special methods of investigation: x-raying, isotopic, endoscopic, morphological, immunological, etc. are of decisive importance.

Different endoscopic methods—investigation of inner organs by means of special optical systems permitting the physician to see, photograph or take material from any inner organ for morphological research—are widely used.

Isotopic methods of investigation are more and more widely introduced into clinical practice. It was stated that some radioactive preparations are absorbed by cancer cells to a greater degree than normal cells. This permits to detect the tumor and their metastases according to radioactive preparation distribution.

Principally new methods of immunologic diagnostics are being used with primary liver cancer and ovary teratoblastoma based on determination of specific embryonal antigen.

Methods of immunological diagnostics (by means of cancer embryonal antigens) are being developed for large intestine, stomach, chorionepithelioma, etc.

Usual laboratory methods (general blood test, urine test, biochemical investigations) do not play decisive role in complex diagnostics of the tumor.

The final stage of diagnostics is morphological investigation. Elucidation of morphological characteristics of tumor structure is very important for choosing the method of treatment.

Tumor treatment is carried out by different methods depending on the tumor kind, its localization, stage of development, age of the patient, etc.

Till today, the oldest and the most widely used method for tumor treatment is surgical intervention.

Besides improvements in technology of surgical interventions, successes in malignant tumor surgery are, to a greater degree, connected with the progress in after-surgery treatment of patients.

Leading scientists and physicians have developed general principles of oncological operations including the idea that healthy tissues surrounding the tumor should be partially removed. Schemes of rational operative interventions for different kinds of tumors have been developed wherein, together with wide excision of the tumor itself, removal of nearest lymphatic nods where probability of metastases is very high is provided for.

In a number of cases surgical interventions are supplemented with radiation and medicinal methods of treatment. Application of radiation methods is based on the principle that tumor cells suffer more from ionized radiation than normal ones. A number of malignant tumors, e.g. cutaneous cancer, lower lip cancer, throat cancer and uterine neck cancer, can be cured at early stages only by means of radiation therapy.

At present radiation therapy is successively used as one of the stages in a complex treatment before or after surgical intervention due to breast cancer, uterine neck cancer, esophagus cancer, throat cancer, soft tissues sarcoma, etc.

Modern methods of radiation therapy are various in relation to the kinds of radiation used, methods of their energy supply to the hotbed of the tumor and apparatuses used. This is a perspective and actively developing direction in oncology.

Methods of medicinal treatment are actively developed. Of principal importance is the proved fact that it is possible to treat (cure) individual oncological patients by means of medicinal preparations.

Experience in using medicines for treatment shows that each of the preparations used in oncological practice is effective only with definite types of tumors,—see for example the results of work Russian Prof. U. Morozov (Rem Petrov “Me or not me”, Moscow, Molodaya Gvardia Publishing House, 1987)

In recent years methods of malignant tumors immunotherapy directed to activating protective forces of patients are being intensively developed.

Due to all the above said, the aim of the proposed invention consists in excluding the main disadvantages of the known methods for cancer treatment irrespective of its kind and the organ targeted, mainly on the basis of early diagnostics of oncological disease (see application for Israel patent No 207661, dated 17 Aug. 2010), i.e. cure of practically any kind of cancer using human immunity without side effects and secondary influence of tumor-forming factors.

### SUMMARY OF THE INVENTION

The aim set forth is attained by the proposed basic variant of cancer cure (treatment) with immediate (direct) contact between proteins secreted by cancer tumor and T-lymphocytes in the patient's blood, including the stage of weakening during some time of ability liquid (humoral) immunity system to work out antibodies with help a medicinal preparation, in particular immunodepressant without weakening of protecting properties of cellular immunity system.

The aim set forth can also be attained by a variant of cancer treatment including stages of direct contact between proteins secreted by cancer tumor and T-lymphocytes of patient's blood effectuated out of the organism (in vitro) with subsequent return of T-lymphocytes into the patient's organism.

The invention proposed represents a real breakthrough in the given field of medicine and with its wide application can bring great profit to mankind in the field of health protection as well as in economy of material and financial resources.

### DETAILED DESCRIPTION OF THE INVENTION

The section given presents explanations of the authentic meaning of the invention and creates breakthrough in the field of medicine which was declared by the author in the division “Summary of the invention”.

According to modern scientific opinions, the main function of immunity is preservation of genetic constancy of the organism inner medium during the whole period of life.

In order to fulfill this function there are two closely interacting immunity systems in the human organism—cellular and humoral (liquid).

Main goals of cellular immune system connected with T-lymphocytes in blood are rejection of foreign tissue and development of immune response against viruses.

Humoral (liquid) system depends on the activity of B-lymphocytes, which, multiplying with stimulation, form a clone of plasmatic cells which synthesize antibodies. Under the influence of antibodies some microbes die and dissolve. Antibodies destroy secreted toxins, poisons and other products of microbe's vital activity.

Usually mutating, i.e. changed cells, appear in the human organism. Their general quantity is higher than a million, and part of them is malignant. Nevertheless, immune system discovers all these cells and destroys them. But sometimes, due to some reasons, this system malfunctions, thus leading to development of cancer tumor.

Even in 1942, English scientist P. Gorer has proved that antibodies against malignant tumors appear in the blood of patients suffering from these tumors. In this case it appeared strange that these antibodies start to protect the tumor instead of fighting against it. This was found due to the following experiments.

G. Snell, Nobel Prize winner, from The Jackson laboratory (Bar-Harbor, Me.) worked at producing pure mouse strains. These mice don't have genetic differences, and, consequently, their organs and tissues are not rejected with transplantation. Together with his pupil N .Callison he found the following regularity. If the mice were inoculated with a malignant tumor by means of cancerogenic substance, corresponding antibodies appeared in their blood. But if this blood is introduced into another mouse, this mouse would be easier inoculated with the same tumor, and it will grow faster. This means that antibodies amplify tumor growth.

This phenomenon was called the phenomenon of immunological amplification.

Swedish immunologists, Karl and Ingrid Hellstrom have developed a method according which T-lymphocytes in a test tube (in vitro) suppressed the growth of malignant cells. Prior to that, scientists developed malignant tumor with mice by means of cancerigenic substance. Tumor cells were put into test tubes with nutrient medium. Then lymphocytes from mice with tumor were added into test-tubes. Growth of malignant cells in tubes was considerably suppressed.

Then scientists have carried out a series of experiments with skin cancer in rabbits. With part of rabbits the tumor was growing leading to the animal death, whereas with the other part of animals tumor decreased up to its complete disappearance.

It was found that lymphocytes in both groups of animals suppress tumor cells growth to a similar degree. But if blood serum with antibodies were added to vessels with malignant cells, the results would be opposite. Serum from the first group suppresses lymphocytes activity, and from the second groups it doesn't.

Scientists have proved that anti-tumor antibodies serve as the serum factor which interferes with lymphocytes ability to suppress malignant cells growth.

According to their conclusion, the destiny of the tumor and tumor-carrier depends on the ratio of antibodies production and storing of immune lymphocytes. In case the first factor prevails, tumor is growing, and in case the second factor prevails, tumor regresses.

So, it is clear that antibodies really interfere with immune system of the organism in fighting malignant tumors.

But in order to understand how antibodies work, let us consider one more experiment.

In 1974, Lea and Boris Cherchic from Manchester have found that before the division starts, living cells undergo body destructuredness which occurs under the influence of strong stimulator of human T-lymphocytes. In 20 minutes destructuredness of cell body was determined, thus certifying lymphocytes activation and their readiness to multiplication. Then the experiment was repeated, but in this case lymphocytes have been taken from people with malignant tumor and activation of T-lymphocytes didn't take place. This means that mechanism of T-lymphocytes stimulation with healthy people and oncological patients is different. And the most important thing found by Cherchic couple is that T-lymphocytes of oncological patients are activated during contact with proteins produced by malignant tumor cells (see Rem Petrov “Me or not me”, Moscow, Molodaya Gvardia Publishing House, 1987, p. 216-217).

The main distinctive feature of all malignant cells consists in the fact that they are divided themselves not obeying the commands and necessities of the whole organism, thus meaning that they differ from all other cells of an individual. Hence, cancer tumor is a hostile tissue for the organism and this, in its turn, means that cellular immunity system should fight such tissues, its main “fighters” being T-lymphocytes. But in case of oncological diseases these lymphocytes in order to be activated should meet proteins secreted by malignant cells.

And it is known that all cancer cells secrete specific antigens which are embryonal proteins. Such proteins are peculiar to in-uterus development and never appear with healthy adults. These proteins being the products of cancer cells vital activity become targets for antibodies which destroy them.

Thus, well-organized interaction of humoral and cellular immunity systems turns into counter-action: humoral (liquid) system destroys proteins without interaction with which T-lymphocytes cannot be activated to destroy cancer cells.

Up to the present time science could not unambiguously explain the higher probability cause of malignant tumor occurrence with elderly people. The author of the proposed invention explains it by the fact that the number of T-lymphocytes in a unit of blood volume is constantly decreasing with age. At the same time the number of B-lymphocytes remains practically constant during the whole lifetime. Their ratio is increasing in favor of the humoral system, that's why anti-cancer protection decreases with age.

This also explains why the phenomenon of immunological amplification appears in such a reliable system of immunity. The matter is that immune system not always is on the side of protecting. During pregnancy it creates the regime of maximum prevalence for the fetus despite the fact that fetus is an antigen for the mother. At that time humoral system acts in the usual way providing reliable anti-infection protection for the mother organism. The fetus as well as cancer cells secreted embryonic proteins into the surrounding. This means that for nature the law of genus continuation is more important than the law of preserving genetic constant in the inner organism medium and, thus, occurrence of embryonal proteins is a signal for immune system switching into another regime of activity, and it is this regime of tissues protection which secretes embryonal proteins.

Due to the above said and on the basis of the results of experiments described earlier, the author of the proposed method according to the present patent application puts forward his approach to treating oncological diseases which consists in the fact that:

for treating oncological diseases it is necessary to decrease the influence of humoral system of immunity on the tumor, thus decreasing the number of antibodies destroying embryonal proteins secreted by cancer cells and creating the possibility of these proteins contact with T-lymphocytes. The result of this contact is activation of T-lymphocytes and destruction of all malignant formations by them irrespective of their location in the organism.

The author consider that the same result will be reached also in the method variant, during which mentioned contact of cancer cells proteins with T-lymphocyts is carriet out outside the patient's organism (invitro), then T-lymphocyts are returned into patient's organism.

As medicinal preparations which can weaken humoral system of immunity for some time without damage to cellular system it is perspective to use special immunodepressants against humoral system (Other approaches are also possible).

At present preparations of opposite action are used during organ transplantations. From the point of view of the approach proposed, these immunodepressants should increase the danger of malignant tumors occurrence as their action is targeted for weakening cellular system of immunity with minimum influence on humoral system.

Practical results completely certify this fear. So, according to the data of American scientists, people with kidney transplant develop cancer 6 times more often than people in the same age group who didn't undergo such an operation.

English journal “Cancer” also certified considerable increase in oncological diseases with patients who have lived more than two years after kidney transplant and were obliged to take corresponding immunodepressant.

The author's statement of the proposed method that weakening the influence of humoral system of immunity on malignant tumor should lead to organism cure is not without proof. Even at the end of the 19th century American physician William B. Coley drew attention to the fact that malignant tumors start regression if oncological patients concurrently get infected with a non-vital illness of bacterial origin. Due to this observation he started treatment of oncological patients by preparations of killed bacteria. This way he succeeded in curing cancer even in very difficult cases. All New-York was talking about the hospital where W. B. Coley worked: even those whose destiny seemed to be fatal, get cured (see V. Vakula “Biotechnology: what is this?”, Moscow, Molodaya Gvardia Printing House, 1989, p. 177) The result was seen, but the mechanism of therapeutic effect was not solved.

Following the logics of the method proposed by the author of the present patent application, it is easy to note that preparations of bacterial origin have attracted part of the forces of humoral system of immunity and this, in its turn, have led to the fact that forces of cellular immunity started to overcome humoral forces. It is this change in the ratio of two systems of immunity when fighting cancer that became the cause of malignant tumor regression.

Analogous approach to cancer treatment has been proposed and already tested on mice by Italian and American scientists from Milan University (see http://science.km.ru/magazin/view print.asp?id). They have introduced (into the tumor) modified salmonella bacteria which do not cause salmonellosis thus caused powerful immune response which destroyed cancer cells.

These modern researches certify the principal possibility of cancer treatment by W. B. Coley method.

So, both variants of the cancer treatment method proposed (including the second variant clearly formulated in the division “Summary of the invention” and disclosed earlier in this division and representing further development of the method proposed by Hellstrom couple) are really attainable and effective in principle.

Either of them can be considered preferable under definite conditions (taking into account other aspects).

Within the framework of the preset application, the author of the proposed invention considers the first variant (on the basis of weakening humoral system of immunity by an immunodepressant) as a preferable one and is determined to further finding or developing a definite, most effective immunodepressant for leading the proposed method for clinical practice.

In a general case, the first variant of the proposed method should be implemented in the following way.

Immunodepressant of humoral system of immunity is took according to the same scheme as immunodepressants used during organ transplantation. Duration of taking depends on the size of tumor and its localization. During treatment constant supervision on tumor regression dynamics is carried out by any of the methods used at present. Medicine is taken up to the time when tumor and metastases (in case they existed) fully disappear.

In accordance with the above said, the author of the proposed invention considers that the principal attainability of the goal set forth by means of the proposed method is sufficiently grounded, as well as the meaning of the breakthrough in this field of medicine—possibility of curing practically any kind of cancer by means of any of the two proposed methods of treatment.

I claim:

1. Method of cancer treatment with direct contact between proteins secreted by cancer tumor and patient's blood T-lymphocytes including the stage of weakening during some time of ability humoral immunity system to work out antibodies with help medicinal preparation, in particular by immunodepressant without weakening of protecting properties of cellular immunity system.

2. Method of cancer treatment including stages
of direct contact between proteins secreted by cancer tumor and patient's blood T-lymphocytes which is carried out outside the patient's organism (in vitro) and
subsequent returning of T-lymphocytes into patient's organism.
